(19) United States
(12) Patent Application Publication
(10) Pub. No.: US 2007/0092486 A1
(43) Pub. Date: Apr. 26, 2007

(54) Glycolated and glycosylated poultry derived therapeutic proteins

(75) Inventor: Kyle D. Yesland, Athens, GA (US)

Correspondence Address: AVIGENICS, INC., 111 RIVERBEND ROAD, ATHENS, GA, 30605, US

(73) Assignee: AviGenics, Inc.

(21) Appl. No.: 11/584,832

(22) Filed: Oct. 23, 2006

Related U.S. Application Data
(60) Provisional application No. 60/729,429, filed on Oct. 21, 2005.

(51) Int. Cl. A01K 67/027; A61K 38/21; A61K 38/19; C12N 5/06

(52) U.S. Cl. 424/85.1; 424/133.1; 424/85.6; 424/85.7; 800/19; 435/349

(57) ABSTRACT

Compositions containing a glycosylated therapeutic amino acid sequence obtained from a transgenic avian wherein the therapeutic amino acid sequence is a glycoprotein and includes a covalently bonded glycol polymer.

### RELATED APPLICATION INFORMATION

This application claims the benefit of U.S. provision patent application No. 60/729,429, filed Oct. 21, 2005, the disclosure of which is incorporated in its entirety herein by reference.

### BACKGROUND OF THE INVENTION

There is a strong need to develop protein delivery technologies that lower the costs of protein therapeutics to patients and healthcare providers. One solution is the development of methods to prolong the circulating half-lives of protein therapeutics in the body. This solution also satisfies the needs and desires of patients for protein therapeutics that are “user-friendly”, e.g., protein therapeutics that do not require frequent administrations, e.g., injections.

Covalent modification of proteins with glycol polymers such as polyethylene glycol (PEG) has proven to be a useful method to extend the circulating half-lives of proteins in the body (Abuchowski et al., 1984; Hershfield, 1987; Meyers et al., 1991). Covalent attachment of glycol polymers to a protein can increase the protein's effective size and reduce its rate of clearance from the body. Glycol polymers such as PEG are commercially available in a variety of sizes (i.e., molecular weights), allowing the circulating half-lives of glycol polymer modified proteins to be tailored for individual indications through use of different size glycol polymers. Other documented in vivo benefits of glycol polymer modification such as PEG modification are an increase in protein solubility, stability (possibly due to protection of the protein from proteases) and a decrease in protein immunogenicity. See, for example, Katre et al., 1987; Katre, 1990. In addition, glycosylation has been shown to enhance the efficacy of protein therapeutics by, for example, increasing the protein's effective size and reducing its immunogenicity and rate of clearance from the body.

### SUMMARY OF THE INVENTION

It is discovered that glycolation (e.g., PEGylation), in combination with glycosylation, of therapeutic proteins produced in avians can produce a synergistic effect in which the efficacy of the proteins are enhanced significantly beyond that which is obtained by just one of either the glycosylation of the therapeutic protein or the glycolation of the therapeutic protein. Therapeutic proteins produced in an avian system can be glycosylated eliminating the need for in vitro glycosylation as would be required, for example, using therapeutic proteins produced in prokaryotic systems, e.g., E. coli.

In one useful aspect, the invention is drawn to compositions which contain a glycosylated therapeutic amino acid sequence obtained from a transgenic avian, such as a transgenic chicken, wherein the therapeutic amino acid sequence is a glycoprotein associated with a glycol polymer. For example, the glycoprotein may be associated with the glycol polymer by a chemical interaction such as ionic bonding or hydrogen bonding. In one particularly useful embodiment, the glycoprotein is covalently bonded to the glycol polymer. In a particularly useful embodiment of the invention, the therapeutic amino acid sequence is an exogenous amino acid sequence. For example, the therapeutic amino acid sequence may be an amino acid sequence endogenous to a human.

In one useful embodiment, the therapeutic amino acid sequence is cytokine. For example, the therapeutic amino acid sequence may be granulocyte colony stimulating factor, interferon alpha, interferon beta, erythropoietin or granulocyte macrophage colony stimulating factor. In one aspect the cytokine is a cytokine endogenous to a human.

In one aspect of the invention, the glycosylation is provided by an oviduct cell of the transgenic avian. For example, the oviduct cell can be a tubular gland cell.

In one embodiment, the invention is drawn to glycosylations being linked to the proteins by linkages provided for in an avian gene expression system. For example, the therapeutic amino acid sequence may be O-glycosylated and/or the therapeutic amino acid sequence may be N-glycosylated.

The invention contemplates the application of any useful glycol polymer for attachment to a poultry derived glycosylated therapeutic protein. For example, the glycol polymer may be a polyalkylene glycol such as a polyethylene glycol and a polypropylene glycol. The invention is not limited to glycol polymers of any particular molecular weight. For example, the glycol polymers may have a molecular weight of about 200 to about 400,000, for example, about 200 to about 20,000.

The invention contemplates the linking of the glycol polymer to the glycosylated protein by any useful chemical bonding methods known in the art. In one embodiment, the glycol polymer is covalently bonded to an amino group of the therapeutic amino acid sequence. In another example, the glycol polymer is covalently bonded to a carboxyl group of the therapeutic amino acid sequence.

In one useful embodiment, the glycosylated therapeutic amino acid sequence obtained from a transgenic avian is a glycoprotein and comprises a glycol polymer covalently bonded to a glycosylation of the therapeutic amino acid sequence. The invention contemplates the linking of the glycol polymer to any component of the glycosylation of the therapeutic amino acid sequence. For example, and without limitation, the invention contemplates the linking of the glycol polymer to n-acetyl-galactosamine, n-acetyl-glucosamine, galactose and/or n-acetyl-neuraminic acid or any other carbohydrate structure which may be present in the glycosylation.

In one embodiment, therapeutic proteins produced in accordance with the present invention are soluble in an aqueous phase or are substantially soluble in an aqueous phase. The therapeutic proteins produced in accordance with the present invention can be nonimmunogenic or have reduce immunogenicity relative to an otherwise identical glycosylated therapeutic that is not glycolated.

### DEFINITIONS AND ABBREVIATIONS

Certain definitions are set forth herein to illustrate and define the meaning and scope of the various terms used to describe the invention herein.

The terms “active ingredient” and “compound of the invention” refer to a poultry derived glycolated-glycosylated protein therapeutic of the invention.

The term “avian” as used herein refers to any species, subspecies or race of organism of the taxonomic class ava, such as, but not limited to chicken, turkey, duck, goose, quail, pheasants, parrots, finches, hawks, crows and ratites including ostrich, emu and cassowary. The term includes the various known strains of Gallus gallus, or chickens, (for example, White Leghorn, Brown Leghorn, Barred-Rock, Sussex, New Hampshire, Rhode Island, Australorp, Minorca, Amrox, California Gray), as well as strains of turkeys, pheasants, quails, duck, ostriches and other poultry commonly bred in commercial quantities. It also includes an individual avian organism in all stages of development, including embryonic and fetal stages. The term “avian” also may denote “pertaining to a bird”, such as “an avian (bird) cell.”

The term “cytokine” as used herein refers to a proteinaceous signalling compound involved in inter-cell communication. Cytokines play a major role in a variety of immunological, inflammatory and infectious diseases. They are also involved in several developmental processes during embryogenesis. Cytokines are produced by a wide variety of cell types, both haemopoietic and non-haemopoietic, and can have effects on nearby cells or cells throughout the organism, sometimes strongly dependent on the presence of other chemicals and cytokines. Cytokines are typically smaller water-soluble proteins, for example, glycoproteins, with a mass of 8-30 kDa.

“Glycolation” refers to the addition of a glycol polymer to a molecule such as the addition of a glycol polymer to a glycosylated poultry derived protein therapeutic. “Glycolated” refers to a substance, such as a glycosylated poultry derived protein therapeutic, to which a glycol polymer has been added.

A “glycol polymer” as used herein refers to any useful alkene, alkane or alkyne (and combinations thereof) polymer glycol. Examples include, without limitation, polypropylene glycol, polyethylene glycol and polybutylene glycol.

The terms “heterologous” and “exogenous” in general refer to a biomolecule such as a nucleic acid or a protein that is not normally found in a certain cell, tissue or other component contained in or produced by an organism. For example, a protein that is heterologous or exogenous to an egg is a protein that is not normally found in the egg.

The term “inf” means interferon.

The term “PEG” means polyethylene glycol.

As used herein a “standard protein therapeutic” is a protein therapeutic that does not contain a poultry derived glycosylation pattern and a glycol polymer. A standard protein therapeutic can be a protein therapeutic containing a poultry derived glycosylation pattern or a glycol polymer.

“Therapeutic protein”, “protein therapeutic”, “pharmaceutical protein” “therapeutic amino acid sequence” each refer to an amino acid sequence which in whole or in part makes up a drug. In one embodiment, a pharmaceutical composition, pharmaceutical formulation or therapeutic composition includes one or more protein therapeutics, pharmaceutical proteins, therapeutic amino acid sequences or therapeutic proteins.

As used herein, a “transgenic avian” is any avian, as defined herein, in which one or more of the cells of the avian contain heterologous nucleic acid introduced by manipulation, such as by transgenic techniques. The nucleic acid may be introduced into a cell, directly or indirectly, by introduction into a precursor of the cell by way of deliberate genetic manipulation, such as by microinjection or by infection with a recombinant retrovirus, for example, injection of a recombinant replication deficient retrovirus into the subgerminal cavity of an avian embryo. Genetic manipulation also includes classical cross-breeding, or in vitro fertilization. The heterologous nucleic acid may be an artificial chromosome or may be integrated within a chromosome of the avian, or it may be extrachromosomally replicating DNA.

As used herein, “treating” or “treating a condition” refers to administering a pharmaceutical composition or pharmaceutical formulation for preventing disease and/or treating disease. To prevent disease refers to prophylactic treatment of a patient who is not yet ill, but who is susceptible to, or otherwise at risk of, a contracting a particular disease. To treat disease or use for therapeutic treatment refers to administering treatment to a patient already suffering from a disease to ameliorate the disease and improve the patient's well being. Thus, treating or treating a condition is the administration to a mammal one or more glycolated-glycosylated poultry derived therapeutic proteins either for therapeutic or prophylactic purposes.

The abbreviation “g” means grams. The abbreviation “ml” means milliliters. The abbreviation “mg” means milligrams. The abbreviation “PEG” means polyethylene glycol. The abbreviation “KDa” means kilodalton. “° C.” means degrees centigrade. The abbreviation “mM” means millimolar. The abbreviation “mU means milliunits.

### DETAILED DESCRIPTION

This invention specifically contemplates the glycolation, for example, PEGylation, of glycosylated therapeutic proteins produced by avians, including without limitation, chicken, turkey, duck, goose, quail, pheasants, parrots, finches, hawks, crows and ratites including ostrich, emu and cassowary. In one particularly useful embodiment, the invention is drawn to glycolation, for example, PEGylation, of glycosylated therapeutic proteins produced in chickens.

Typically, the genetic sequence present in the host organism determines, with respect to the amino acid sequence of the protein, the location and general structure of the carbohydrate groups. Carbohydrate groups are commonly attached to asparagine, serine or threonine. Methods to produce glycosylated therapeutic proteins useful to produce therapeutic proteins as disclosed herein are known in the art and are described in, for example, U.S. patent application Ser. No. 10/463,980, filed Jun. 17, 2003 (US patent publication No. 2004/0019923) and U.S. patent application Ser. No. 11/068,155, filed Feb. 28, 2005 (US patent publication No. 2006/0015960).

One glycol polymer that is particularly useful in accordance with the present invention is polyethylene glycol (PEG). PEG is a hydrophilic, biocompatible and non-toxic polymer of general formula H (OCH₂CH₂) nOH, wherein n>4. Its molecular weight can vary substantially, for example, from 200 to 20,000 Dalton. The invention is not specifically drawn to any particular method of attaching PEG molecules to the therapeutic proteins or any particular molecular weight of PEG employed.

Many useful methods of glycolating proteins are known in the art and the present invention contemplates the employment of each such method. For example, the invention contemplates any useful method of PEGylation to produce therapeutic proteins as disclosed herein. In one example, certain well known methods for PEGylating proteins use compounds such as N-hydroxysuccinimide (NHS)-PEG to attach PEG to free amines, typically at lysine residues or at the N-terminal amino acid. Some such methods can PEGylate the therapeutic protein in a non-site specific manner, which in certain instances may not be preferred.

Site specific methods of PEGylation are also included in the present invention. One such method attaches PEG to cysteine residues using cysteine-reactive PEGs. A number of highly specific, cysteine-reactive PEGs with different reactive groups (e.g., maleimide, vinylsulfone) and different size PEGs (2-40 kDa) are commercially available. At neutral pH, these PEG reagents selectively attach to “free” cysteine residues, i.e., cysteine residues not involved in disulfide bonds. Through in vitro mutagenesis using recombinant DNA techniques, additional cysteine residues can be introduced at any useful position in the protein. The newly added “free” cysteines can serve as sites for the specific attachment of a PEG molecule using cysteine-reactive PEGs. The added cysteine residue can be a substitution for an existing amino acid in a protein, added preceding the amino-terminus of the protein or after the carboxy-terminus of the protein, or inserted between two amino acids in the protein. Alternatively, one of two cysteines involved in a native disulfide bond, which may be present in certain therapeutic proteins, may be deleted or substituted with another amino acid, leaving a native cysteine (the cysteine residue in the protein that normally would form a disulfide bond with the deleted or substituted cysteine residue) free and available for chemical modification. In one embodiment, the amino acid substituted for the cysteine would be a neutral amino acid such as serine or alanine. In addition, disulfide bonds can be reduced and alkylated with iodoacetimide without impairing biological activity providing targets for deletion or substitution by another amino acid.

In one embodiment, methods for preparing a glycolated, for example, PEGylated glycoprotein comprise the steps of (a) reacting the protein with polyethylene glycol (such as a reactive ester or aldehyde derivative of PEG) under conditions whereby the protein becomes attached to one or more PEG groups and (b) obtaining the reaction product(s). In general, the optimal reaction conditions for the reactions will be determined case by case based on known parameters and the desired result.

There are a number of attachment methods available to those skilled in the art. See, for example, EP 0 401 384, the disclosure of which is hereby incorporated by reference; see also, Malik et al. (1992), Exp. Hematol., 20:1028-1035; Francis (1992), Focus on Growth Factors, 3(2):4-10, (published by Mediscript, Mountain Court, Friem Barnet Lane, London N20 OLD, UK); EP 0 154 316; EP 0 401 384; WO 92/16221; WO 95/34326; and the other publications cited herein that relate to addition of a glycol polymer to a protein (e.g., PEGylation) the disclosures of which are hereby incorporated by reference.

In one embodiment, glycol polymer molecules such as polyethylene glycol polymer molecules can be “activated” to facilitate coupling of the glycol polymer molecule to the avian or poultry derive glycosylated therapeutic protein. Examples of preparation of such activated glycol polymers are provided in the following references which are hereby incorporated by reference: K. Yoshinaga and J. M. Harris, J. Bioact. Comp. Polym., 1, 17-24 (1989); K. Nilsson and K. Mosbach, Methods in Enzymology, 104, 56 (1984); C. Delgado, G. E. Francis, and D. Fisher, in “Separations Using Aqueous Phase Systems,” D. Fisher and I. A. Sutherland, Eds., Plenum, London, 1989, pp. 211-213; M.-B. Stark and J. K. Holmberg, Biotech. Bioeng., 34, 942 (1989); J. M. Harris and K. Yoshinaga, J. Bioact. Compat. Polym., 4, 281 (1989); H. Walter, D. E. Brooks, and D. Fisher (Editors), “Partitioning in Aqueous Two-Phase Systems,” Academic Press, Orlando, Fla., 1985; D. Fisher and I. A. Sutherland (Editors), “Separations Using Aqueous Phase Systems: Applications in Cell Biology and Biotechnology,” Plenum, London, 1989.

U.S. Pat. No 4,002,531, issued Jan. 11, 1977, the disclosure of which is incorporated in its entirety herein by reference, describes preparation of PEG acetaldehyde for attaching PEG to enzymes and other proteins. Such methods are contemplated for the attachment of PEG to glycosylated therapeutic proteins.

U.S. Pat. No 4,179,337, issued Dec. 18, 1979, the disclosure of which is incorporated in its entirety herein by reference, discloses certain methods for attaching PEG to proteins to provide soluble PEG-protein conjugates. Such methods are contemplated for the attachment of PEG to glycosylated therapeutic proteins.

Glycolation such as PEGylation may be carried out by, for example, an acylation reaction or an alkylation reaction with a reactive or activated polyethylene glycol polymer molecule. Thus, protein products produced according to the present invention include PEGylated proteins wherein the PEG group(s) is (are) attached by acyl or alkyl groups. Such products may be mono-PEGylated or poly-PEGylated (e.g., containing 2-6, and/or 2-5, PEG groups). The PEG groups can be attached to the protein at the alpha or epsilon amino groups of amino acids, but it is also contemplated that the PEG groups could be attached to any group attached to the protein which is sufficiently reactive to become attached to a PEG group under suitable reaction conditions.

Glycolation such as PEGylation by acylation generally can involve reacting an active ester derivative of glycol polymer such as PEG with the protein. For the acylation reactions, the polymer(s) selected can have a single reactive ester group. Any known or subsequently discovered reactive PEG molecule may be used to carry out the PEGylation reaction. A useful activated PEG ester is PEG esterified to N-hydroxysuccinimide (NHS). As used herein, “acylation” is contemplated to include, without limitation, the following types of linkages between the therapeutic protein and a glycol polymer such as PEG: amide, carbamate, urethane, and the like (Chamow (1994), Bioconjugate Chem., 5 (2): 133-140). Reaction conditions may be selected from any of those known in the PEGylation art or those subsequently developed, but should avoid conditions such as temperature, solvent and pH that would inactivate the therapeutic poultry derived protein to be modified.

Glycolation by acylation will generally result in a poly-PEGylated protein. In one embodiment, the connecting linkage is an amide. Also, the resulting product may be substantially only (e.g., >95%) mono, di- or tri-PEGylated. However, some species with higher degrees of PEGylation may be formed in amounts depending on the specific reaction conditions used. If desired, more purified PEGylated species may be separated from the mixture (particularly unreacted species) by standard purification techniques, including among others, dialysis, salting-out, ultrafiltration, ion-exchange chromatography, gel filtration chromatography and electrophoresis.

Glycolation such as PEGylation by alkylation can involve reacting a terminal aldehyde derivative of a glycol polymer such as PEG with the protein in the presence of a reducing agent. For the reductive alkylation reaction, the polymer(s) selected can have a single reactive aldehyde group. An exemplary reactive PEG aldehyde is polyethylene glycol propionaldehyde, which is water stable, or mono C1-C10 alkoxy or aryloxy derivatives thereof. See, for example, U.S. Pat. No. 5,252,714, issued Oct. 12, 1993, the disclosure of which is incorporated in its entirety herein by reference.

Glycolation such as PEGylation by alkylation can also result in poly-PEGylated protein. In addition, one can manipulate the reaction conditions to substantially favor glycolation only at the alpha amino group of the N-terminus of the protein to provide a mono-PEGylated protein. In either case the glycol polymer groups are often attached to the protein by a —CH₂—NH-group.

Reductive alkylation to produce a substantially homogeneous population of mono-polymer/protein product can include the steps of:

(a) reacting a poultry derived glycosylated therapeutic protein with a reactive PEG molecule under reductive alkylation conditions, at a pH suitable to permit selective modification of the alpha amino group at the amino terminus of the protein; and

(b) obtaining the reaction product(s). Derivatization by reductive alkylation to produce a monoPEGylated product.

The reaction can be performed at a pH which allows one to take advantage of the pKa differences between the epsilon amino groups of the lysine residues and that of the alpha amino group of the N-terminal residue of the protein. In general, if the pH is lower, a larger excess of polymer to protein will be desired (i.e., the less reactive the N-terminal alpha amino group, the more polymer needed to achieve optimal conditions). If the pH is higher, the polymer:protein ratio need not be as large (i.e., more reactive groups are available, so fewer polymer molecules are needed). In one embodiment, the pH can fall within the range of 3 to 9, for example, 3 to 6. For the reductive alkylation, the reducing agent should be stable in aqueous solution and preferably be able to reduce only the Schiff base formed in the initial process of reductive alkylation. Suitable reducing agents may be selected from sodium borohydride, sodium cyanoborohydride, dimethylamine borane, trimethylamine borane and pyridine borane. A particularly suitable reducing agent is sodium cyanoborohydride. Other reaction parameters such as solvent, reaction times, temperatures and means of purification of products can be determined on a case-by-case basis, based on the published information relating to derivatization of proteins with water soluble polymers.

By such selective derivatization, attachment of a glycol polymer that contains a reactive group such as an aldehyde to a protein is controlled. The conjugation with the polymer takes place predominantly at the N-terminus of the protein and no significant modification of other reactive groups, such as the lysine side chain amino groups, occurs. The preparation can typically be greater than 90% monopolymer/protein conjugate, or greater than 95% monopolymer/protein conjugate, with the remainder of observable molecules being unreacted (i.e., protein lacking the polymer moiety).

Glycolation also may be carried out by water soluble polymers having at least one reactive hydroxy group (e.g. polyethylene glycol) that can be reacted with a reagent having a reactive carbonyl, nitrile or sulfone group to convert the hydroxyl group into a reactive Michael acceptor, thereby forming an activated linker useful in modifying various proteins to provide improved biologically-active conjugates. Reactive carbonyl, nitrile or sulfone means a carbonyl, nitrile or sulfone group to which a two carbon group is bonded having a reactive site for thiol-specific coupling on the second carbon from the carbonyl, nitrile or sulfone group. See, for example, WO 92/16221, the disclosure of which is incorporated in its entirety herein by reference).

The activated linkers can be monofunctional, bifunctional, or multifunctional. Useful reagents having a reactive sulfone group that can be used in the methods include, without limitation, chlorosulfone, vinylsulfone and divinylsulfone.

In a specific embodiment, the glycol polymer is activated with a Michael acceptor. WO 95/13312, the disclosure of which is incorporated in its entirety herein by reference, describes, among other things, water soluble sulfone-activated PEGs which are highly selective for coupling with thiol moieties instead of amino moieties on molecules and on surfaces. These PEG derivatives are stable against hydrolysis for extended periods in aqueous environments at pHs of about 11 or less, and can form linkages with molecules to form conjugates which are also hydrolytically stable. The linkage by which the PEGs and the biologically active molecule are coupled includes a sulfone moiety coupled to a thiol moiety and has the structure PEG--SO₂—CH₂—CH₂—S—W, where W represents the biologically active molecule, and wherein the sulfone moiety can be vinyl sulfone or an active ethyl sulfone. Two useful homobifunctional derivatives are PEG-bis-chlorosulfone and PEG-bis-vinylsulfone.

In one particularly useful embodiment, the glycosylated therapeutic protein is glycolated (e.g., PEGylated) by the coupling of a glycol polymer to the glycosylated therapeutic protein through glycosylations present on the protein. Therefore, the invention includes glycosylated protein therapeutics having glycol polymers such as polyethylene glycol coupled to a glycosylation structure of the glycosylated therapeutic protein and methods of making such glycosylated-glycolated protein therapeutics.

In one specific embodiment contemplated for use in the present invention, the invention is drawn to a process for the glycolation of a glycosylated macromolecule, comprising activating a polyalkylene glycol, reacting the activated polyalkylene glycol with a diamino compound, whereby the activated polyalkylene glycol is coupled to the diamino compound through one of its amino groups, oxidizing a poultry derived glycosylated therapeutic protein to activate at least one glycosyl group therein, and reacting the polyalkylene glycol coupled to the diamino compound with the oxidized glycosyl group in the macromolecule. For example, the invention can include a process for the PEGylation of a glycosylated macromolecule comprising:
(a) reacting a polyethylene glycol of the formula
   CH₃O—(CH₂CH₂O)ₙ—H with
   o-nitrophenylchloroformate and triethylamine to produce a nitro compound of the formula CH₃O—(CH₂CH₂O)ₙ—COO-Ph-NO₂,
(b) reacting the nitro compound with a diaminoalkane of the formula H₂N—(CH₂)ₓ—NH₂ to produce an amino compound of the formula CH₃O—(CH₂CH₂O)ₙ—CO—NH—(CH₂)ₙ—NH₂,
(c) oxidizing sugar groups on the avian derived glycosylated therapeutic protein to produce a macromolecule with an oxidized sugar residue, and
(d) reacting the amino compound with the activated macromolecule to produce a PEGylated molecule. In one embodiment, the molecular weight of the polyethylene glycol is up to about 24,000; and accordingly n is about 2 to about 500. In this embodiment, the diaminoalkane, x is typically about 1 to about 20.

The result of this preferred process is a PEGylated glycosylated avian derived glycosylated therapeutic protein, wherein PEG is bonded to the protein through its glycosylations, specifically, of the formula PEG-OCO—NH-alkylene-N═CH-avian derived glycosylated therapeutic protein. Other aspects of this method of glycolating glycosylated therapeutic proteins of the invention are disclosed in WO 94/05332, published Mar. 17, 1994, the disclosure of which is incorporated in its entirety herein by reference.

The invention can be used to produce a wide range of desired glycolated and glycosylated therapeutic proteins such as fusion proteins, growth hormones, cytokines, structural proteins and enzymes including human growth hormone, interferon, lysozyme, and β-casein. Other possible proteins contemplated for modification as disclosed herein include, but are not limited to, albumin, α-1 antitrypsin, antithrombin III, collagen, factors VIII, IX, X (and the like), fibrinogen, hyaluronic acid, insulin, lactoferrin, protein C, erythropoietin (EPO), granulocyte colony-stimulating factor (G-CSF), granulocyte macrophage colony-stimulating factor (GM-CSF), tissue-type plasminogen activator (tPA), somatotropin, and chymotrypsin. Modified immunoglobulins and antibodies, including immunotoxins which bind to surface antigens on human tumor cells and destroy them, can also be produced as disclosed herein.

Other specific examples of therapeutic proteins which are contemplated for combined glycolation and glycosylation include, without limitation, factor VIII, b-domain deleted factor VIII, factor VIIa, factor IX, anticoagulants; hirudin, alteplase, tpa, reteplase, tpa, tpa −3 of 5 domains deleted, insulin, insulin lispro, insulin aspart, insulin glargine, long-acting insulin analogs, hgh, glucagons, tsh, follitropin-beta, fsh, gm-csf, pdgh, ifn alpa2a, inf-apha, inf-beta 1b, ifn-beta 1a, ifn-gamma1b, il-2, il-11, hbsag, ospa, murine mab directed against t-lymphocyte antigen, murine mab directed against tag-72, tumor-associated glycoprotein, fab fragments derived from chimeric mab, directed against platelet surface receptor gpII(b)/III(a), murine mab fragment directed against tumor-associated antigen ca125, murine mab fragment directed against human carcinoembryonic antigen, cea, murine mab fragment directed against human cardiac myosin, murine mab fragment directed against tumor surface antigen psma, murine mab fragments (fab/fab2 mix) directed against hmw-maa, murine mab fragment (fab) directed against carcinoma-associated antigen, mab fragments (fab) directed against nca 90, a surface granulocyte nonspecific cross reacting antigen, chimeric mab directed against cd20 antigen found on surface of b lymphocytes, humanized mab directed against the alpha chain of the il2 receptor, chimeric mab directed against the alpha chain of the il2 receptor, chimeric mab directed against tnf-alpha, humanized mab directed against an epitope on the surface of respiratory synctial virus, humanized mab directed against her 2, i.e., human epidermal growth factor receptor 2, human mab directed against cytokeratin tumor-associated antigen anti-ctla4, chimeric mab directed against cd 20 surface antigen of b lymphocytes dornase-alpha dnase, beta glucocerebrosidase, tnf-alpha, il-2-diptheria toxin fusion protein, tnfr-lgg fragment fusion protein laronidase, dnaases, alefacept, darbepoetin alfa (colony stimulating factor), tositumomab, murine mab, alemtuzumab, rasburicase, agalsidase beta, teriparatide, parathyroid hormone derivatives, adalimumab (lggl), anakinra, biological modifier, nesiritide, human b-type natriuretic peptide (hbnp), colony stimulating factors, pegvisomant, human growth hormone receptor antagonist, recombinant activated protein c, omalizumab, immunoglobulin e (lge) blocker, lbritumomab tiuxetan, ACTH, glucagon, somatostatin, somatotropin, thymosin, parathyroid hormone, pigmentary hormones, somatomedin, erythropoietin, luteinizing hormone, chorionic gonadotropin, hypothalmic releasing factors, antidiuretic hormones, prolactin and thyroid stimulating hormone. In one embodiment, the invention is drawn to the production of poultry derived glycolated-glycosylated human proteins, such as the human form (i.e., endogenous to a human) of the proteins disclosed herein.

The invention contemplates the modification of immunoglobulins and other multimeric proteins as disclosed herein. Examples of therapeutic antibodies that may be modified in methods of the invention include but are not limited to HERCEPTINTM (Trastuzumab) (Genentech, Calif.) which is a humanized anti-HER2 monoclonal antibody for the treatment of patients with metastatic breast cancer; REOPRO™ (abciximab) (Centocor) which is an anti-glycoprotein IIb/IIIa receptor on the platelets for the prevention of clot formation; ZENAPAX™ (daclizumab) (Roche Pharmaceuticals, Switzerland) which is an immunosuppressive, humanized anti-CD25 monoclonal antibody for the prevention of acute renal allograft rejection; PANOREX™ which is a murine anti-17-IA cell surface antigen IgG2a antibody (Glaxo Wellcome/Centocor); BEC2 which is a murine anti-idiotype (GD3 epitope) IgG antibody (ImClone System); IMC-C225 which is a chimeric anti-EGFR IgG antibody (ImClone System); VITAXIN™ which is a humanized anti-αVβ3 integrin antibody (Applied Molecular Evolution/MedImmune); Campath 1H/LDP-03 which is a humanized anti CD52 IgG1 antibody (Leukosite); Smart M195 which is a humanized anti-CD33 IgG antibody (Protein Design Lab/Kanebo); RITUXAN™ which is a chimeric anti-CD2O IgG1 antibody (IDEC Pharm/Genentech, Roche/Zettyaku); LYMPHOCIDE™ which is a humanized anti-CD22 IgG antibody (Immunomedics); ICM3 is a humanized anti-ICAM3 antibody (ICOS Pharm); IDEC-114 is a primate anti-CD80 antibody (IDEC Pharm/Mitsubishi); ZEVALIN™ is a radiolabelled murine anti-CD20 antibody (IDEC/Schering AG); IDEC-131 is a humanized anti-CD40L antibody (IDEC/Eisai); IDEC-151 is a primatized anti-CD4 antibody (IDEC); IDEC-152 is a primatized anti-CD23 antibody (IDEC/Seikagaku); SMART anti-CD3 is a humanized anti-CD3 IgG (Protein Design Lab); 5G1.1 is a humanized anti-complement factor 5 (CS) antibody (Alexion Pharm); D2E7 is a humanized anti-TNF-α antibody (CATIBASF); CDP870 is a humanized anti-TNF-α Fab fragment (Celltech); IDEC-151 is a primatized anti-CD4 IgG1 antibody (IDEC Pharm/SmithKline Beecham); MDX-CD4 is a human anti-CD4 IgG antibody (Medarex/Eisai/Genmab); CDP571 is a humanized anti-TNF-α IgG4 antibody (Celltech); LDP-02 is a humanized anti-α4β7 antibody (LeukoSite/Genentech); OrthoClone OKT4A is a humanized anti-CD4 IgG antibody (Ortho Biotech); ANTOVA™ is a humanized anti-CD40L IgG antibody (Biogen); ANTEGREN™ is a humanized anti-VLA-4 IgG antibody (Elan); and CAT-152, a human anti-TGF-β₂ antibody (Cambridge Ab Tech).

In one embodiment, the therapeutic protein contemplated for modification as disclosed herein is an antibody capable of selectively binding to an antigen which may be generated by combining at least one immunoglobulin heavy chain variable region and at least one immunoglobulin light chain variable region, for example, cross-linked by at least one disulfide bridge. The combination of the two variable regions generates a binding site that binds an antigen using methods for antibody reconstitution that are well known in the art.

While it is possible that, for use in therapy, proteins of the invention may be administered in raw form, it is preferable to administer the protein as part of a pharmaceutical formulation.

The invention thus further provides a pharmaceutical formulation comprising a poultry derived glycosylated-glycolated therapeutic protein or a pharmaceutically acceptable derivative thereof together with one or more pharmaceutically acceptable carriers thereof and, optionally, other therapeutic and/or prophylactic ingredients. The carrier(s) must be “acceptable” in the sense of being compatible with the other ingredients of the formulation and not deleterious to the recipient thereof.

Pharmaceutical formulations include those suitable for oral, rectal, nasal, topical (including buccal and sub-lingual), vaginal or parenteral (including intramuscular, sub-cutaneous and intravenous) administration or in a form suitable for administration by inhalation or insufflation. The formulations may, where appropriate, be conveniently presented in discrete dosage units and may be prepared by any of the methods well known in the art of pharmacy. All methods include the step of bringing into association the active compound with liquid carriers or finely divided solid carriers or both and then, if necessary, shaping the product into the desired formulation.

Pharmaceutical formulations suitable for oral administration may conveniently be presented as discrete units such as capsules, cachets or tablets each containing a predetermined amount of the active ingredient; as a powder or granules; as a solution; as a suspension; or as an emulsion. The active ingredient may also be presented as a bolus, electuary or paste. Tablets and capsules for oral administration may contain conventional excipients such as binding agents, fillers, lubricants, disintegrants, or wetting agents. The tablets may be coated according to methods well known in the art. Oral liquid preparations may be in the form of, for example, aqueous or oily suspensions, solutions, emulsions, syrups or elixirs, or may be presented as a dry product for constitution with water or other suitable vehicle before use. Such liquid preparations may contain conventional additives such as suspending agents, emulsifying agents, non-aqueous vehicles (which may include edible oils) or preservatives.

The compounds according to the invention may also be formulated for parenteral administration (e.g., by injection, for example bolus injection or continuous infusion) and may be presented in unit dose form in ampoules, pre-filled syringes, small volume infusion or in multi-dose containers with an added preservative. The compositions may take such forms as suspensions, solutions, or emulsions in oily or aqueous vehicles, and may contain formulatory agents such as suspending, stabilizing and/or dispersing agents. Alternatively, the active ingredient may be in powder form, obtained by aseptic isolation of sterile solid or by lyophilization from solution, for constitution with a suitable vehicle, e.g., sterile, pyrogen-free water, before use.

For topical administration to the epidermis, the compounds according to the invention may be formulated as ointments, creams or lotions, or as a transdermal patch. Ointments and creams may, for example, be formulated with an aqueous or oily base with the addition of suitable thickening and/or gelling agents. Lotions may be formulated with an aqueous or oily base and will in general also contain one or more emulsifying agents, stabilizing agents, dispersing agents, suspending agents, thickening agents or coloring agents.

Formulations suitable for topical administration in the mouth include lozenges comprising active ingredient in a flavored base, usually sucrose and acacia or tragacanth; pastilles comprising the active ingredient in an inert base such as gelatin and glycerin or sucrose and acacia; and mouthwashes comprising the active ingredient in a suitable liquid carrier.

Pharmaceutical formulations suitable for rectal administration wherein the carrier is a solid, are most preferably represented as unit dose suppositories. Suitable carriers include cocoa butter and other materials commonly used in the art, and the suppositories may be conveniently formed by a mixture of the active compound with the softened or melted carrier(s) followed by chilling and shaping in molds.

Formulations suitable for vaginal administration may be presented as pessaries, tampons, creams, gels, pastes, foams or sprays containing in addition to the active ingredient, such carriers as are known in the art to be appropriate.

For intra-nasal administration the compounds of the invention may be used as a liquid spray or dispersible powder or in the form of drops.

Drops may be formulated with an aqueous or non-aqueous base also comprising one or more dispersing agents, solubilizing agents or suspending agents. Liquid sprays are conveniently delivered from pressurized packs.

For administration by inhalation, the compounds according to the invention are conveniently delivered from an insufflator, nebulizer or a pressurized pack or other convenient means of delivering an aerosol spray. Pressurized packs may comprise a suitable propellant such as dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount.

Alternatively, for administration by inhalation or insufflation, the compounds according to the invention may take the form of a dry powder composition, for example a powder mix of the compound and a suitable powder base such as lactose or starch. The powder composition may be presented in unit dosage form in, for example, capsules or cartridges or, e.g., gelatin or blister packs from which the powder may be administered with the aid of an inhalator or insufflator.

When desired, the above described formulations adapted to give sustained release of the active ingredient, may be employed.

The pharmaceutical compositions according to the invention may also contain other active ingredients such as antimicrobial agents, or preservatives.

In addition, it is contemplated that the compounds of the invention may be used in combination with other therapeutic agents. For example, poultry derive glycosylated-glycolated human interferon alpha (e.g., interferon alpha 2b) can be used in combination with ribavirin and/or virimidine to treat viral infections such as hepatitis C.

Compositions or compounds of the invention can be used to treat a variety of conditions. For example, there are many conditions for which treatment therapies are known to practitioners of skill in the art in which protein therapeutics are employed. The present invention contemplates that the protein therapeutics produced in an avian system resulting in a poultry derived glycosyation pattern and thereafter glycolated in accordance with the present invention can be employed to treat such conditions. That is, the invention contemplates the treatment of a condition known to be treatable by a protein therapeutic having a certain amino acid sequence by administering a protein therapeutic having the same certain amino acid sequence which is produced in an avian system and is glycosylated and glycolated.

It is specifically contemplated that the glycosylated-glycolated therapeutic proteins produced in accordance with the present invention will require a reduced frequency of administration and/or a reduced dosage of therapeutic protein relative to the frequency of administration and/or dosage required to treat a condition utilizing the same protein therapeutic not having the avian glycosylation and glycolation (i.e., a standard protein therapeutic). For example, a dosage of glycosylated-glycolated therapeutic protein of the invention may be employed that equals about 10% or about 20% or about 30% or about 40% or about 50% or about 60% or about 70% or about 80% of the dosage typically employed to treat a condition or precondition using the same protein therapeutic not having the avian glycosylation and glycolation (i.e., standard protein therapeutic). The frequency of administration of a glycosylated-glycolated therapeutic protein of the invention may be reduced by, for example, about 10% or about 20% or about 30% or about 40% or about 50% or about 60% or about 70% or about 80% relative to the frequency of administration of the same protein therapeutic not having the avian glycosylation and glycolation (i.e., standard protein therapeutic).

Generally, the dosage administered will vary depending upon known factors such as age, health and weight of the recipient, type of concurrent treatment, frequency of treatment, and the like. Usually, a dosage of active ingredient can be between about 0.0001 and about 10 milligrams per kilogram of body weight. Precise dosage, frequency of administration and time span of treatment can be determined by a physician skilled in the art of therapeutic protein administration.

The following examples are methods that may be particularly useful for the production of glycosylated and glycolated protein therapeutics in accordance with the present invention; however, it is understood that the invention is not limited to any particular method of making therapeutic proteins of the invention and the invention encompasses all such useful methods known in the art and those yet to be devised.

### EXAMPLE 1

### Preparation of Gylcosylated-PEGylated Poultry Derived Human Ervthropoietin

### Preparation of PEG-4-hydroxy-6-chloro-1 3,5-triazine (PEG-HTA)

30 g of PEG 750 (about 0.04 mole) or 80 g of PEG 2,000 (about 0.04 mole) is dissolved in 150 ml anhydrous benzene containing 8 g Na₂CO₃. The solution is cooled to 10° C. and 7.38 g cyanuric chloride is added. The solution is stirred overnight at 10° C. Five ml of water is added and the solution then is brought to room temperature for several hours, followed by heating at 40° C. overnight. Insoluble material is removed by centrifugation and the solvent is removed by reduced pressure in a rotary evaporator at 40° C. A small amount of precipitate which sometimes appears during concentration is removed by the addition of a small amount of benzene to lower the viscosity, followed by centrifugation and reconcentration. The PEG-3-hydroxy-6-chloro-1,3,5-triazine, a viscous liquid at 40° C. is stored in the freezer.

### Preparation of PEG-HTA-EPO Conjugate

Poultry derived glycosylated EPO is produced as disclosed in U.S. Pat. No. 6,730,822, issued May 4, 2004, the disclosure of which is incorporated in its entirety herein by reference. 10 mg of the EPO is dissolved in 1 ml 0.1 M borate buffer, pH 9.2 and 179 mg of PEG-HTA 2,000 is added. After 2 hours unreacted PEG-HTA is removed by passing the solution through a column of Sephadex G-10. The PEG-HTA-EPO conjugate is concentrated on a rotary evaporator and is stored in the freezer.

In accordance with the foregoing procedure, but using PEG-750, a similar product can be obtained. In accordance with the foregoing procedure, but carrying out the reaction at pH 8.5 and at pH 10, similar products can be obtained.

### EXAMPLE 2

### Preparation of O-PEG-(P-azo Poultry Derived Glycosylated Interferon Alpha 2-b benzyl)ether

### Formation of O-PEG-p-amino benzyl ether

3.46 g of p-Nitrobenzyl chloride, 2.0 g of powdered sodium hydroxide, 20 ml of anhydrous tetrahydrofuran and 0.01 mole of PEG are refluxed for 3 hours. The solution is filtered and evaporated under reduced pressure and PEG-p-nitronenzyl ether is precipitated by the addition of petroleum ether (bp 30° C. to 40° C). The nitro ether is reduced with hydrogen at atmospheric pressure in the presence of Raney nickel catalyst (about 1 g) in ethanol (50 ml). The catalyst is removed and the filtrate evaporated yielding O-PEG-p-amino benzyl ether.

### Coupling with Interferon Alpha 2-b

O-PEG-p-amino benzyl ether is diazotized in aqueous solution at 0° C. with nitrous acid. To the purified diazotized solution an aqueous solution of 0.25% glycosylated interferon alpha 2b, produced as disclosed in U.S. Pat. No. 6,730,822, issued May 4, 2004, is added and the mixture is kept at 0° C. for 2 hours. The solution is dialysed at 5° C. to 10° C. to yield glycosylated-PEGylated interferon alpha 2b.

### EXAMPLE 3

### Preparation of O-PEG Methyl Carboxy Poultry Derived Human GM-CSF

### Preparation of PEG-Methyl Carbomethoxy Ester

2.0 g of PEG 750 is dissolved in 30 ml of liquid ammonia and the solution is treated with sodium until a blue color persists for 5 minutes. The ammonia is allowed to evaporate on a stream of dry nitrogen. The residue is treated with 5 ml of methyl chloroacetate and the mixture is allowed to stand overnight at room temperature followed by heating to 100° C. for 1 hour. The excess reagent is removed under reduced pressure to provide PEG-methyl carbomethoxy ester.

### Activation of PEG

To a solution of 1.0 g of O-PEG-methyl carbomethoxy ester in 10 ml water a solution of 1.0 g of N-ethoxycarbonyl-2-ethoxy 1,2-dihydroquinoline (EEDQ) in 10 ml of 10% acetone is added dropwise. The pH is maintained at 7.0 and after 30 minutes, the pH is adjusted to 1.0 with concentrated hydrochloric acid and is maintained at this pH for 90 seconds to destroy excess EEDQ. The pH of the solution is then adjusted to pH 8.

### Coupling to GM-CSF

50 mg of human glycosylated GM-CSF produced as disclosed in U.S. Pat. No. 6,730,822, issued May 4, 2004, in phosphate buffer, pH 8.0, is added to the solution of the activated PEG at 4° C. to 5° C. After ½ hour the solution is dialyzed against water yielding glycosylated-pegylated poultry derived GM-CSF.

### EXAMPLE 4

### Preparation of 1-(Poultry Derived Human G-CSF-2-hydroxy propoxy)-4-3−-O-PEG-2″-hydroxy Propoxy Butane

### Oxirane Ether of PEG

5.0 g of PEG, 1 ml of 1,4-butanediol diglycidyl ether and 1 ml of 0.6 M sodium hydroxide solution containing 2 mg of sodium borohydride are stirred at room temperature for 8 hours. The solution is neutralized and evaporated. The residue is extracted with acetone and the PEG ether precipitated by the addition of excess petroleum ether.

### Coupling to G-CSF

1.0 g of oxirane-PEG and 50 mg of human G-CSF produced as disclosed in U.S. Pat. No. 6,730,822, issued May 4, 2004, in buffer solution (pH 8.5) are allowed to react at room temperature for 48 hours. The solution is dialyzed to yield poultry derived glycosylated-PEGylated G-CSF.

### EXAMPLE 5

### Preparation of PEGylated, Glycosylated Poultry Derived Human Interferon beta 1a

### Activation of Methoxy-PEG (MPEG)

Two grams of 15 KDa mPEG (0.1 mM, final concentration), is dissolved in 20 ml of acetonitrile with 0.24 g of o-nitrophenylchloroformate (1.2 mM) and 33 microliters of triethylamine (1.2 mM) and is stirred for 24 hours at room temperature.

The triethylammonium chloride is then filtered off using a sintered glass funnel. 200 ml of ethyl ether is added, and the solution is left to crystallize overnight at 4° C. The product is filtered, washed with ether to remove all of the yellow color, and recrystallized from acetonitrile-ether. The product is then assayed spectrophotometrically by the release of p-nitrophenol by ε-amino-n-caproic acid (ACA).

### PEGylation of Poultry Derived Human Interferon Beta 1a through Lysine Groups

5 mg of poultry derived human Interferon beta I a, produced as disclosed in U.S. Pat. No. 6,730,822, issued May 4, 2004, is dialyzed extensively into 50 mM sodium borate buffer pH 8.3.

To the 2 ml dialyzed sample 3 mg of the activated mPEG is added, a 5 molar excess. Immediately, 3 mg of activated mPEG is added and incubated at room temperature with shaking for a further 30 minutes. The reaction is stopped after 2 hours; final molar excess is 20-fold, by loading the sample on a NAP 25 (Pharmacia) desalting column and eluting it with 50 mM NaPO₄ buffer, pH 6.8. The desalted sample is loaded on Superose 6 column (1×30 cm BioRad Econocolumn®) and eluted with 50 mM NaPO₄ buffer, pH 6.8. Resultant peaks from the Superose column are assayed by SDS-PAGE and pooled.

### EXAMPLE 6

### Preparation of Poultry Derived Human Interferon Beta 1a Having PEG Coupled to a Glycosylation Structure Present on the Interferon

### Making the Amino Derivative of mPEG-μ-pNP (PEG-μ-butamine)

0.5 g of mPEG-μ-p-nitrophenyl is slowly added to 5 ml of 50 mM Na-borate buffer, pH 9.0, containing 44.25 mg (100 mmoles) of 1,4-aminobutane. The reaction is incubated at room temperature with shaking for 3 hours. The reaction is stopped by passing it through an NAP 25 desalting column and eluted with water and dialyzed into milli-Q H₂O. The dialyzed material is lypophylized and weighed.

### Oxidation of Poultry Derived Human Interferon Beta 1a

| | | |
|---|---|---|
| Coupling Buffer: | 0.05 | M sodium acetate |
| | 0.1 | M sodium chloride, pH 5.0 |
| Wash Buffer: | 0.1 | M sodium acetate |
| | 0.5 | M sodium chloride, pH 3.5 |
| Storage Buffer: | 0.05 | M sodium phosphate, pH 6.8 |

0.5 mg of poultry derived human interferon beta la is buffer exchanged into the coupling buffer using an NAP-10 (Pharmacia) desalting column. To the poultry derived protein solution is added 0.1 ml of freshly prepared 100 mM sodium m-periodate (NaIO4). The solution is mixed gently, and the sealed reaction vessel is shielded from light and incubated at room temperature for 30 minutes. To stop the reaction, the sample is passed through a NAP-10 desalting column and is equilibrated with wash buffer. The column is eluted with the conjugation buffer.

### Coupling of Oxidized Poultry Derived Human Interferon Beta 1a to PEG-μ-butamine

To the desalted, oxidized poultry derived human interferon beta 1 a is added 5 mg of PEG-μ-butamine. The reaction mix is overlayed with nitrogen and is tumbled gently overnight at 4° C. The molar ratio of poultry derived human interferon beta 1a to PEG-μ-butamine is 1:100. The sample is then loaded following optional reduction of the poultry derived human interferon beta 1a onto a Superose 6 column. The interferon containing peaks are pooled and are concentrated on an amicon stirred cell concentrator.

### EXAMPLE 7

### Preparation of O-linked 40 Kilodalton PEG Linked to Poultry Derived Human Antibody using ST3Ga1III

### Desialylation

In this step poultry derived glycosyalted human antibody is desialylated. The GlcNAc-Gal linkage serves as an acceptor for transfer of the modified sialic acid PEG.

Poultry derived glycosylated human antibody solution 10 ml (0.33 μmol) is buffer exchanged with Tris buffer (20 mM Tris, 50 mM NaCl, 5 mM CaCl₂, 0.02% NaN₃, pH 7.2) to give a final volume of 10 ml. Then 750 mU 2,3,6,8-neuramidase, from Arthrobacter Ureafaciens, is added to the solution. The resulting mixture is rocked at 32° C. for 48 hours.

### O-Linked PEGylation

In this step O-sialyltranferase is used to transfer a modified sialic acid-PEG moiety to the desialylated poultry derived glycosylated human antibody. CMP-sialic acid-PEG (40 KDa, 33 mg, 0.825 μmol), O-sialyltransferase (1.4 U/ml, 300 mU), and 0.25 mL of 100 mM MnCl₂ are added to the above mixture. The mixture is then rocked at 32° C. for 48 hours. After the 48 hour period, the reaction mixture is concentrated by ultrifiltration (MWCO 5K) to 2.8 ml, then buffer exchanged with 25 mM NaOAc+0.001% Tween-80, pH 6.0, to a final volume of 3 ml. The final product is ion exchange purified. PEGylated poultry derived glycosyalted human antibody is collected and concentrated by ultrifiltration.

### Complete Terminal Sialylation of CHO— Poultry Derived Glycosyalted Human Antibody-Galnac-Gal-SA-PEG

In this step of the process sialic acid is added to the termini of glycosyl structures not bearing a modified sialic acid residue.

Combined PEGylated poultry derived glycosyalted human antibody (approximately 2 mg) is concentrated by ultrifiltration (MWCO 5K) and then buffer exchanged with tris buffer (0.05M Tris, 0.15 M NaCl, 0.001 M CaCl₂+0.005% NaN₃) to a final volume of 2 ml, then CMP-N-acetyl neuraminic acid (CMP-NANA; 1.5 mg, 2.4 μmol), ST3Ga;1III (8.9 U/ml, 10 μl, 0.098 U) and 50 μl of 1100 mM MnCl₂ are added. The resulting mixture is rocked at 32° C. for 24 h, then concentrated to 1 ml final volume. This solution is directly subjected to Superdex 200 purification.

What is claimed is:

1. A composition containing a glycosylated therapeutic amino acid sequence obtained from a transgenic avian wherein the therapeutic amino acid sequence is a glycoprotein and comprises a covalently bonded glycol polymer.

2. The composition of claim 1 wherein the avian is a chicken.

3. The composition of claim 1 wherein the therapeutic amino acid sequence is an exogenous amino acid sequence.

4. The composition of claim 1 wherein the therapeutic amino acid sequence is an amino acid sequence endogenous to a human.

5. The composition of claim 1 wherein the therapeutic amino acid sequence is cytokine.

6. The composition of claim 1 wherein the therapeutic amino acid sequence is selected from the group consisting of granulocyte colony stimulating factor, interferon alpha, interferon beta, erythropoietin and granulocyte macrophage colony stimulating factor.

7. The composition of claim 6 wherein the therapeutic amino acid sequence is an amino acid sequence endogenous to a human.

8. The composition of claim 6 wherein the therapeutic amino acid sequence is an antibody.

9. The composition of claim 1 wherein the glycosylation is provided by an oviduct cell of the transgenic avian.

10. The composition of claim 9 wherein the oviduct cell is a tubular gland cell.

11. The composition of claim 1 wherein the therapeutic amino acid sequence is O-glycosylated.

12. The composition of claim 1 wherein the therapeutic amino acid sequence is N-glycosylated.

13. The composition of claim 1 wherein the glycol polymer is a polyalkylene glycol.

14. The composition of claim 1 wherein the glycol polymer is a polyethylene glycol.

15. The composition of claim 1 wherein the glycol polymer is a polypropylene glycol.

16. The composition of claim 1 wherein the glycol polymer has a molecular weight of about 300 to about 200,000.

17. The composition of claim 1 wherein the glycol polymer is covalently bonded to an amino group of the therapeutic amino acid sequence.

18. The composition of claim 1 wherein the glycol polymer is covalently bonded to a carboxyl group of the therapeutic amino acid sequence.

19. A composition containing a glycosylated therapeutic amino acid sequence obtained from a transgenic avian wherein the therapeutic amino acid sequence is a glycoprotein and comprises a glycol polymer covalently bonded to a glycosylation of the therapeutic amino acid sequence.

20. A composition containing a glycosylated therapeutic amino acid sequence obtained from a transgenic chicken wherein the therapeutic amino acid sequence is a glycoprotein and comprises a covalently bonded glycol polymer.
